(19) United States
(12) Patent Application Publication
(10) Pub. No.: US 2025/0208030 A1
(43) Pub. Date: Jun. 26, 2025

(54) OPTICAL DEVICE FOR LASER EMISSION SPECTROSCOPIC ANALYSIS, LASER EMISSION SPECTROSCOPIC ANALYZER, LASER EMISSION SPECTROSCOPIC ANALYSIS METHOD, AND MOLTEN METAL PLATING EQUIPMENT

(71) Applicant: NIPPON STEEL CORPORATION, Tokyo (JP)

(72) Inventors: Norihiro TSUJI, Tokyo (JP); Michihiro AIMOTO, Tokyo (JP); Tomonori AOKI, Tokyo (JP); Hideki NISHIMURA, Tokyo (JP)

(73) Assignee: NIPPON STEEL CORPORATION, Tokyo (JP)

(21) Appl. No.: 18/844,764

(22) PCT Filed: Mar. 16, 2023
(86) PCT No.: PCT/JP2023/010393

§ 371 Date: Sep. 6, 2024

(30) Foreign Application Priority Data
Mar. 16, 2022 (JP) 2022-041009

(51) Int. Cl. G01N 21/25; G01N 21/27; G01N 33/205; G01N 33/208

(52) U.S. Cl. CPC G01N 21/255; G01N 21/27; G01N 33/205; G01N 33/208; G01N 2201/0231; G01N 2201/06113

(57) ABSTRACT

To enable lightweight, compact, and sufficient analytical precision.

An optical device for laser emission spectroscopic analysis of the present invention includes: a casing unit that has a laser oscillator oscillating laser light, a condenser lens condensing the laser light and on which the laser light emitted from the laser oscillator is directly incident, and an optical fiber light receiver receiving light emitted from plasma generated by irradiating the laser light onto molten metal at a light-receiving end surface and guiding the light to an exit-side end surface; and a cylindrical probe that is connected to the casing unit so that a center axis is parallel to an oscillation axis of the laser light in the laser oscillator, supplies inert gas to an opening end located downstream of the laser light traveling direction, and guides the laser light toward the opening end to irradiate on the molten metal, wherein a surface normal direction at the light-receiving end surface of the optical fiber light receiver is parallel to the oscillation axis of the laser light.

### TECHNICAL FIELD

The present invention relates to an optical device for laser emission spectroscopic analysis, a laser emission spectroscopic analyzer, a laser emission spectroscopic analysis method, and a molten metal plating equipment.

### BACKGROUND ART

For molten metal baths such as hot-dip galvanizing baths, it is necessary to monitor and control constituents to control quality of products (for example, hot-dip galvanized steel sheets, and other products) obtained using such molten metal baths and to control operating conditions.

For example, in a hot-dip galvanizing manufacturing process, trace amounts of Al and Fe are added to the hot-dip galvanizing bath to optimize a plating film and a steel sheet and to improve anticorrosive effect. During operation, Al tends to decrease and Fe tends to increase as the steel sheet passes through the hot-dip galvanizing bath.

When contents of trace elements in the hot-dip galvanizing bath deviate from an appropriate range, it may cause plating defects, or these trace elements may alloy with zinc to form dross, which may hinder the operation. Therefore, it is important to control the contents of these trace elements by continuously analyzing them and to add trace elements to keep them within the appropriate range or remove the dross formed.

As a method for analyzing the contents of such trace elements, Patent Document 1 below proposes a method and device for laser emission spectroscopic analysis of molten metals. In addition, Patent Document 2 below discloses a method and device in which a laser-induced breakdown spectroscopy (LIBS) is applied to the analysis of molten materials.

### PRIOR ART DOCUMENT

### Patent Document

Patent Document 1: Japanese Laid-open Patent Publication No. 2006-300819

Patent Document 2: Translation of PCT International Application Publication No. 2005-530989

### DISCLOSURE OF THE INVENTION

### Problems to Be Solved by the Invention

However, the devices proposed in Patent Documents 1 and 2 above are both large in size and heavy in weight. Therefore, it requires a great deal of labor to detach, convey, and attach the device to change an observation position of the molten metal bath, for example. In addition, there may be restrictions on the attachment position of the device depending on a configuration of the molten metal bath. For example, even if a conventional device configuration was downsized, the size limit was 500 mm (width)×250 mm (height)×640 mm (depth).

The present invention was made in view of the above problems, and an object of the present invention is to provide an optical device for laser emission spectroscopic analysis, a laser emission spectroscopic analyzer, a laser emission spectroscopic analysis method, and a molten metal plating equipment that are lightweight, compact, and capable of achieving sufficient analytical precision.

### Means for Solving the Problems

To solve the above problems, the present inventors have studied diligently and found that, it is possible to significantly reduce the weight and size of the device while maintaining sufficient measurement precision by omitting as much as possible an optical system for guiding laser light oscillated from a laser oscillator in a desired state, an optical system for condensing light emitted from plasma generated by irradiating the laser light to an object when the light emitted from the plasma is received, and the like, which have been considered essential in the past.

The gist of the invention completed based on such findings is as follows.

(1) An optical device for laser emission spectroscopic analysis, which is used for analyzing components of molten metal, including: a casing unit that has a laser oscillator oscillating laser light, a condenser lens condensing the laser light and on which the laser light emitted from the laser oscillator is directly incident, and an optical fiber light receiver receiving light emitted from plasma generated by irradiating the laser light onto the molten metal at a light-receiving end surface; and a cylindrical probe that is connected to the casing unit so that a center axis is parallel to an oscillation axis of the laser light in the laser oscillator, supplies inert gas to an opening end located downstream of the laser light traveling direction and guides the laser light toward the opening end to irradiate on the molten metal, wherein a surface normal direction at the light-receiving end surface of the optical fiber light receiver is parallel to the oscillation axis of the laser light.

(2) The optical device for laser emission spectroscopic analysis according to (1), wherein the cylindrical probe is connected to the casing unit so that the center axis is coaxial with the oscillation axis of the laser light.

(3) The optical device for laser emission spectroscopic analysis according to (1) or (2), wherein the condenser lens is provided at a connection part between the casing unit and the cylindrical probe.

(4) The optical device for laser emission spectroscopic analysis according to any one of (1) to (3), wherein at least a part of the light emission is incident on the light-receiving end surface of the optical fiber light receiver in a state where the light is not condensed.

(5) The optical device for laser emission spectroscopic analysis according to any one of (1) to (4), wherein the laser oscillator is a diode-pumped laser oscillator.

(6) The optical device for laser emission spectroscopic analysis according to any one of (1) to (5), wherein the condenser lens has an antireflection film on its surface to prevent reflection of the laser light.

(7) The optical device for laser emission spectroscopic analysis according to any one of (1) to (6), further including an angle adjustment mechanism that adjusts a lens optical axis direction of the condenser lens by changing an attachment angle of the condenser lens.

(8) A laser emission spectroscopic analyzer, including: the optical device for laser emission spectroscopic analysis according to any one of (1) to (7); a spectral optical unit that spectrally disperses the light emission guided by the optical fiber light receiver; a detector that detects the light emission spectrally dispersed by the spectral optical unit; and a component analysis unit that analyzes components of molten metal based on detection results of the light emission by the detector.

(9) The laser emission spectroscopic analyzer according to (8), wherein the detector is an image intensifier charge-coupled device detector.

(10) The laser emission spectroscopic analyzer according to (8) or (9), further including: a cooling mechanism that cools an inside of the casing unit.

(11) A laser emission spectroscopic analysis method, which analyzes molten metal in a plating bath for molten metal plating by using the laser emission spectroscopic analyzer according to any one of (8) to (10).

(12) The laser emission spectroscopic analysis method according to (11), wherein the molten metal plating is hot-dip galvanizing.

(13) A molten metal plating equipment, including the laser emission spectroscopic analyzer according to any one of (8) to (10).

(14) The molten metal plating equipment according to (13), which is a hot-dip galvanizing equipment to apply hot-dip galvanizing.

### Effect of the Invention

As explained above, the present invention makes it possible to provide an optical device for laser emission spectroscopic analysis, a laser emission spectroscopic analyzer, a laser emission spectroscopic analysis method, and a molten metal plating equipment that are lightweight, compact, and capable of achieving sufficient analytical precision.

### BRIEF DESCRIPTION OF THE DRAWINGS

FIG. 1 is an explanatory diagram schematically illustrating an example of a configuration of an optical device for laser emission spectroscopic analysis according to an embodiment of the present invention.

FIG. 2 is an explanatory diagram schematically illustrating another example of the configuration of the optical device for laser emission spectroscopic analysis according to the embodiment.

FIG. 3 is a block diagram schematically illustrating an example of a configuration of a laser emission spectroscopic analyzer according to the embodiment.

FIG. 4 is a block diagram illustrating an example of a configuration of an arithmetic processing unit held by the laser emission spectroscopic analyzer according to the embodiment.

FIG. 5 is a block diagram illustrating an example of a hardware configuration of the arithmetic processing unit according to the embodiment.

FIG. 6 is an explanatory diagram schematically illustrating an example of a hot-dip galvanizing equipment as an example of a molten metal plating equipment according to the embodiment.

FIG. 7 is a laser-induced breakdown spectrum illustrating signal strength of Al and Zn for a hot-dip galvanizing bath measured in examples.

FIG. 8 is a graphic diagram illustrating time course of Al concentration in the hot-dip galvanizing bath measured in examples.

### EMBODIMENTS FOR CARRYING OUT THE INVENTION

Hereinafter, a detailed description of a preferred embodiment of the present invention will be explained with reference to the accompanying drawings. In this specification and the drawings, components that have substantially the same functional configuration will be omitted from the duplicated explanation by applying the same codes.

### Optical Device for Laser Emission Spectroscopic Analysis

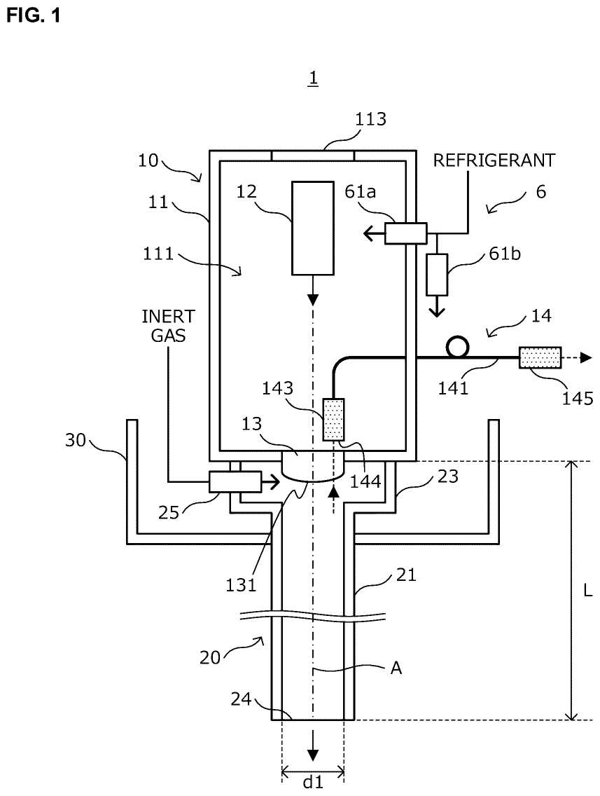

First, a detailed description of an optical device for laser emission spectroscopic analysis according to an embodiment of the present invention will be given with reference to FIG. 1. FIG. 1 is an explanatory diagram schematically illustrating an example of a configuration of the optical device for laser emission spectroscopic analysis of this embodiment.

An optical device 1 for laser emission spectroscopic analysis illustrated in FIG. 1 is an optical device used to analyze components of an object (more precisely, molten metal) and is a device used as a measurement probe for a laser emission spectroscopic analyzer to perform so-called laser-induced breakdown spectroscopy (LIBS).

The optical device 1 for laser emission spectroscopic analysis supplies inert gas from a cylindrical probe 20, irradiates pulsed laser light oscillated by a laser oscillator 12 toward the object, and receives the light (hereinafter referred to as plasma light) emitted from plasma generated at an interface between the object and the inert gas.

As illustrated in FIG. 1, the optical device 1 for laser emission spectroscopic analysis has a casing unit 10, the cylindrical probe 20, and a hood 30.

As illustrated in FIG. 1, the casing unit 10 has a casing 11, the laser oscillator 12, a condenser lens 13, and an optical fiber light receiver 14.

The casing 11 is a casing, and its internal space 111 houses the laser oscillator 12 and at least a light-receiving part 143 of the optical fiber light receiver 14. The casing 11 has a longitudinal direction that is almost parallel to a laser oscillation axis A in this embodiment, and the condenser lens 13 is fixed at a tip side of the casing 11. On the other hand, the casing 11 has an opening part 113 at its base end side, and the opening part 113 functions as a working space for maintenance of the laser oscillator 12, as well as a passage for not-illustrated wiring, and the like. Furthermore, the opening part 113 also functions as an outlet for refrigerant by a cooling mechanism 40, which will be described later.

The casing 11 can be made of known materials such as various resin materials or metal materials. The casing 11 is smaller than a casing of a conventional optical device for laser emission spectroscopic analysis for the reasons described below.

The laser oscillator 12 is a device that oscillates pulsed laser light (hereinafter simply referred to as “laser light”). Since the laser oscillator 12 is required to function as an illumination light source for LIBS analysis, the laser oscillator 12 must have the ability to oscillate pulsed laser light capable of evaporating an object of component analysis (for example, each component of Zn, Fe, Al, or other components that make up a hot-dip galvanizing bath, which is an example of molten metal) without selectivity. As such a laser oscillator 12, various types of pulsed laser light sources that oscillate high-power pulsed laser that enables LIBS analysis can be used. Such pulsed laser light sources include, for example, solid-state lasers such as ruby lasers, Ti sapphire lasers, and YAG lasers, gas lasers such as CO₂ gas lasers, Ar ion lasers, He—Ne ion lasers, excimer lasers, various semiconductor lasers, fiber lasers, and other lasers.

In the optical device 1 for laser emission spectroscopic analysis of this embodiment, a diode-pumped laser oscillator is particularly preferably used as the laser oscillator 12. The diode-pumped laser oscillator is an oscillator with excellent stability of one pulse, which is oscillated. Since LIBS performs the analysis for each pulse, the stability of the laser light for each pulse is improved, resulting in further improvement of analytical precision.

In addition, the diode-pumped laser oscillators make it possible to reduce a frequency of maintenance of the oscillator compared to generally used flashlamp-pumped laser oscillators. In the optical device 1 for laser emission spectroscopic analysis of this embodiment, the laser light oscillated from the laser oscillator 12 is configured to be directly incident on the condenser lens 13, which is described below, so the laser oscillation axis A must be strictly adjusted each time maintenance is performed. The use of a diode-pumped laser oscillator reduces the frequency of maintenance and the frequency of adjustment of the laser oscillation axis A. This not only improves convenience for the user but also suppresses variations in measurement precision caused by variations in the laser oscillation axis A.

The laser oscillator 12 is arranged in the casing 11 so that the laser oscillation axis A of the laser light is almost parallel to a center axis of the cylindrical probe 20. As a result, in this embodiment, the laser light oscillated from the laser oscillator 12 is guided directly to the cylindrical probe 20 without using a conventionally used light-guiding optical system such as a laser reflection mirror. The laser oscillation axis A of the laser light is more preferably arranged to be almost coaxial with the center axis of the cylindrical probe 20. This enables the laser light oscillated from the laser oscillator 12 to be reliably guided to the object without being wasted and dissipated.

In general, a position and angle of the laser oscillation axis are extremely important in the laser emission spectroscopic analysis, and it has conventionally been considered essential to use the light-guiding optical system such as the laser reflection mirror to adjust the position and angle of the laser oscillation axis. In particular, when analyzing molten metal in a molten metal plating bath, and the like, analysis at a relatively deep position in the bath is required, and the cylindrical probe tends to be relatively long to prevent deterioration and damage to the device due to high temperature of the molten metal. In such cases, considering a possibility that the laser oscillation axis may deviate and the laser light may be unwillingly irradiated onto the cylindrical probe, it is generally considered that the importance of the light-guiding optical system, such as the laser reflection mirror, is greater than in the case of analyzing other objects. Contrary to such conventional common-sense recognition by those skilled in the art, the inventors have found that in a device with a relatively long cylindrical probe for molten metal or other objects, the position and angle of the laser oscillation axis can be set to a degree that does not cause problems in measurement even if the light-guiding optical system, such as the laser reflection mirror, is omitted. Thus, it is possible to reduce the size and weight of the optical device for laser emission spectroscopic analysis by omitting the light-guiding optical system such as the laser reflection mirror.

The laser oscillator 12 may be fixed to the casing 11 by a fixing member (not illustrated) that allows the position of the laser oscillator 12 to be adjusted. This allows the position of the laser oscillator 12 to be adjustable and the position and angle of the laser oscillation axis to be adjustable.

The condenser lens 13 is arranged on the laser oscillation axis L on the tip side of the casing 11, and the laser light emitted from the laser oscillator 12 is directly incident on a lens surface. The condenser lens 13 condenses the laser light emitted from the laser oscillator 12 and guides the laser light to the cylindrical probe 20.

The laser light condensed by the condenser lens 13 is generally required to be focused near an opening end 24 of the cylindrical probe 20 described below, or preferably further forward in a traveling direction of the laser light than the opening end 24.

The condenser lens 13 of this embodiment is provided at an interface with the cylindrical probe 20 at the casing 11 (or it can be regarded as a connection part of the cylindrical probe 20), as illustrated in FIG. 1, while using a not-illustrated O-ring or the like in combination. This allows the condenser lens 13 to function as a light-guide window that separates the internal space 111 of the casing 11 and a space inside the cylindrical probe 20 and also prevents inert gas supplied to the cylindrical probe 20 from entering the internal space 111 of the casing 11. By having the condenser lens 13 play multiple roles as described above, an arrangement of an additional light-guide window can be omitted, and the optical device 1 for laser emission spectroscopic analysis can be made even lighter.

The condenser lens 13 may be a lens group formed of a plurality of lenses. However, the condenser lens 13 is preferably formed of a single lens from a viewpoint of further weight reduction of the optical device 1 for laser emission spectroscopic analysis.

The condenser lens 13 may be fixed to the casing 11 by a fixing member (not illustrated) that allows adjustment of an attachment angle of the condenser lens 13. Such a fixing member functions as an angle adjustment mechanism to adjust an optical axis direction of the lens of the condenser lens 13, enabling the position and angle of the laser oscillation axis to be adjusted. This enables fine adjustment of the laser oscillation axis and further improvement of measurement precision, even when the laser light oscillated from the laser oscillator 12 is directly incident on the condenser lens 13.

An antireflection film to prevent reflection of the laser light is preferably provided on at least a surface 131 on the cylindrical probe 20 side of the condenser lens 13. This prevents the laser light reflected on the surface 131 of the condenser lens 13 from reaching the laser oscillator 12 directly and damaging the laser oscillator 12.

As such antireflection films, various known antireflection films can be used, including, for example, dielectric multilayer films, in which dielectric films are multilayered.

The optical fiber light receiver 14 receives the light emission (plasma light) emitted from the plasma generated by irradiating the laser light onto an object at a light-receiving end surface. The optical fiber light receiver 14 has a bundle fiber 141, which is a bundle of optical fibers, the light-receiving part 143, and an emitting part 145, as illustrated in FIG. 1.

The light-receiving part 143 is connected to one end of the bundle fiber 141, and at least the light-receiving part 143 of the optical fiber light receiver 14 is housed inside the casing 11. In the optical device 1 for laser emission spectroscopic analysis of this embodiment, the light-receiving part 143 is provided so that a surface normal direction at the light-receiving end surface 144 of the light-receiving part 143 is parallel to the laser oscillation axis A, as schematically illustrated in FIG. 1. This causes at least a part of the plasma light to enter the light-receiving end surface 144 of the light-receiving part 143 in a state where the light is not condensed.

Here, “the surface normal direction of the light-receiving end surface 144 is parallel to the laser oscillation axis A” shall include not only the case where the surface normal direction and the laser oscillation axis A are completely parallel but also the case where the surface normal direction is inclined with respect to the laser oscillation axis A by a certain allowable angle. The inventors have found that when the angle between the surface normal direction and the laser oscillation axis A is within 10°, enough plasma light to perform LIBS analysis with the required precision can be received.

The closer the angle between the surface normal direction and the laser oscillation axis A is to parallel, the more plasma light can be detected. The angle between the surface normal direction and the laser oscillation axis A is preferably 1.0° or less, and more preferably 0.6° or less.

The plasma light generated by irradiating the laser light on an object is very weak. For this reason, it has conventionally been considered necessary to condense the plasma light using a condensing optical system formed by lenses, and the like, to detect as much of the generated plasma light as possible to perform analysis with high precision. However, the inventors have found that the condensing optical system can be omitted and the plasma light is incident on the light-receiving end surface 144 of the light-receiving part 143 without being condensed. This makes it possible to significantly reduce the weight and size of the optical device 1 for laser emission spectroscopic analysis.

In this embodiment, the plasma light received by the light-receiving end surface 144 of the light-receiving part 143 is a part of the overall plasma light generated because it is not condensed. However, when the plasma light is condensed and received, any deviation of the optical axis of the laser light will result in a larger variation of signal strength obtained. For this reason, the inventors have found that in this embodiment, where only a part of the plasma light is received without being condensed, effects of thermal distortion, vibration, and bath level fluctuation of a molten metal plating device on analysis results can be even suppressed.

In the optical fiber light receiver 14, the bundle fiber 141 transmits the plasma light received at the light-receiving end surface 144 of the light-receiving part 143 to the emitting part 145. The emitting part 145 is connected to a spectral optical unit to be described below when the optical device 1 for laser emission spectroscopic analysis is incorporated into a laser emission spectroscopic analyzer 2.

As illustrated in FIG. 1, the light-receiving part 143 is preferably arranged near the condenser lens 13 so as not to block the laser light oscillated from the laser oscillator 12. By arranging the light-receiving part 143 near the condenser lens 13, it is possible to receive more plasma light and further improve the analysis precision.

The cylindrical probe 20 is a cylindrical member having a probe part 21 and a base end part 23 that is provided on a base end side of the probe part 21 and fixes the probe part 21 to the casing 11.

The base end part 23 has a larger diameter than the probe part 21, supports the probe part 21 on a tip side, and is fixed to the casing 11 on the base end side. An inert gas inlet (gas inlet) 25 is arranged on a side surface of the base end part 23, and inert gas is supplied to a hollow portion of the cylindrical probe 20 through this gas inlet 25.

The probe part 21 is a cylindrical member that forms an optical path between the object to be analyzed and the laser oscillator 12 and is arranged so that the opening end 24 is open to the object. When the object is molten metal, a tip portion of the probe part 21 is immersed in the molten metal. The pulsed laser light irradiated from the laser oscillator 12 is guided by the probe part 21 and focused near the opening end 24 (more preferably, on a forward side of the laser light traveling direction than the opening end 24).

Inert gas is supplied to the probe part 21 from the gas inlet 25 toward the opening end 24 and is emitted from the opening end 24 toward the object. The laser light guided through the probe part 21 is irradiated onto the object, generating plasma at an interface between the inert gas and the object.

An inert gas supply mechanism is not limited, and any known mechanisms such as an inert gas source and supply pipes can be used. The inert gas supplied is preferably the inert gas commonly used in plasma emission spectrometry, such as Ar or He.

The cylindrical probe 20 is a hollow member whose cross-section (more precisely, a cross-section when cut orthogonal to a center axis) has various shapes, such as square, polygonal, circular, and elliptical. In the cylindrical probe 20 of this embodiment, a size of the cross-section may not be constant along a direction of its center axis. For example, the size of the cylindrical probe 20 may be thicker on the laser oscillator 12 side and thinner on the tip side, or thinner on the laser oscillator 12 side and thicker on the tip side. However, considering the fact that the laser light is guided by the hollow portion, the cylindrical probe 20 is preferably cylindrical in shape.

The cylindrical probe 20 is connected to the casing unit 10 such that its center axis is parallel to the laser oscillation axis A of the laser light oscillated from the laser oscillator 12, and more preferably, such that its center axis is coaxial with the laser oscillation axis A of the laser light oscillated from the laser oscillator 12. Here, “the center axis of the cylindrical probe 20 is parallel to the laser oscillation axis A” shall include not only the case where the center axis of the cylindrical probe 20 is perfectly parallel to the laser oscillation axis A but also the case where the center axis is inclined by a certain allowable angle with respect to the laser oscillation axis A. In addition, “the center axis of the cylindrical probe 20 is coaxial with the laser oscillation axis A” shall include not only the case where the center axis and the laser oscillation axis A are perfectly aligned but also the case where the center axis is inclined to some extent with respect to the laser oscillation axis A.

Concretely, in the above “parallel” or “coaxial” mode, the allowable range of the angle between the laser oscillation axis A and the center axis of the cylindrical probe 20 is, when an effective opening diameter of the cylindrical probe 20 to the object is Deff (cm) and a distance from the laser oscillator 12 to the object is LL (cm), limited to arctan (Deff/LL) or less. For example, when Deff and LL are 1.5 cm and 120 cm, respectively, the angle between the center axis of the cylindrical probe 20 and the laser oscillation axis A is limited to 0.36° or less.

Regarding the coaxiality of the laser oscillation axis A and the center axis of the cylindrical probe 20, a maximum distance between an extension line of the center axis of the cylindrical probe 20 at a surface of the condenser lens 13 on the laser oscillator 12 side and each intersection point of the laser light is within Deff (cm), more preferably within 0.1×Deff (cm).

The effective opening diameter is a diameter of the largest circle centered at a point where the center axis of the cylindrical probe 20 intersects the object at the opening end 24 of the cylindrical probe 20 on the object side, not including a solidified material adhered on an inner wall of the cylindrical probe 20. This allows the laser light emitted by the laser oscillator 12 to be irradiated onto the object through the cylindrical probe 20 without the use of a conventionally used laser reflection mirror.

In general, a shorter length of the cylindrical probe is preferable from viewpoints of ease of the optical axis adjustment of the laser light and detection efficiency of the plasma light. However, when the object to be analyzed is a high-temperature substance such as molten metal, each component of the optical device 1 for laser emission spectroscopic analysis is affected by radiant heat or the like from the molten metal, which can affect the analysis precision. Considering both the ease of optical axis adjustment and detection efficiency of plasma light and the effect on the analysis precision caused by the radiant heat, or the like, the length of the cylindrical probe 20 (length L in FIG. 1) is preferably 60 cm or more and 150 cm or less. This makes it possible to achieve both the ease of the optical axis adjustment and the detection efficiency of the plasma light and prevention of degradation of the analytical precision. The length L of the cylindrical probe 20 is more preferably 70 cm or more and 300 cm or less, and even more preferably 80 cm or more and 150 cm or less.

A diameter of the probe part 21 (d1 in FIG. 1) is preferably, for example, 1.5 cm or more and 5.0 cm or less, and more preferably 2.5 cm or more and 3.5 cm or less. This will more reliably prevent an optical path of the laser light from colliding with the inner wall of the cylindrical probe 20, even when the length L of the cylindrical probe 20 is relatively long, as described above.

The inner wall near the opening end 24 of the cylindrical probe 20 may be covered with solidified or adhered substances such as molten metal, originating from the object to be analyzed. When the laser light is irradiated onto such solidified or adhered substances, analysis may become difficult. Therefore, the optical axis is preferably controlled so that an irradiation point of the laser light to the object is near the center axis of the cylindrical probe 20 (in other words, an installation position of the laser oscillator 12 should be adjusted).

The hood 30 is provided to cover the base end part 23 of the cylindrical probe 20 and the casing unit 10 when viewed from the tip side of the cylindrical probe 20. By covering the base end part 23 of the cylindrical probe 20 and the casing unit 10, the hood 30 can suppress the effect of radiant heat from the object on the casing unit 10. The hood 30 also functions as a flow path for refrigerant (for example, cooling gas, and the like) supplied from a cooling mechanism 6 described below.

As explained above, the optical device 1 for laser emission spectroscopic analysis of this embodiment is configured so that the laser oscillation axis A of the laser oscillator 12 and the center axis of the cylindrical probe 20 are almost coaxial, and the surface normal direction at the light-receiving end surface 144 of the light-receiving part 143 is almost parallel to the laser oscillation axis A. This makes it possible to omit conventionally required components such as a laser reflection mirror used to adjust the optical axis of the laser light oscillated from the laser oscillator and an optical member used to condense the plasma light. As a result, the optical device 1 for laser emission spectroscopic analysis is lightweight and compact.

The optical device 1 for laser emission spectroscopic analysis of this embodiment can be used to analyze any object, but molten metal, which is relatively high temperature, is preferred as an object. Since molten metal is relatively high temperature and has large temperature irregularities and changes in temperature at a measurement site, it is desirable to measure at a relatively deep position or at multiple positions to obtain reliable analysis results. Since the optical device 1 for laser emission spectroscopic analysis of this embodiment is lightweight and compact, it is easy to move the optical device 1 for laser emission spectroscopic analysis to perform measurements at multiple measurement sites.

As mentioned above, the optical system 1 for laser emission spectroscopic analysis is suitable for analyzing devices that handle molten metal, for example, a plating bath in a molten metal plating equipment. The molten metal stored in the plating bath includes, for example, molten zinc, molten aluminum, and other molten metals.

### Modification Example of Optical Device for Laser Emission Spectroscopic Analysis

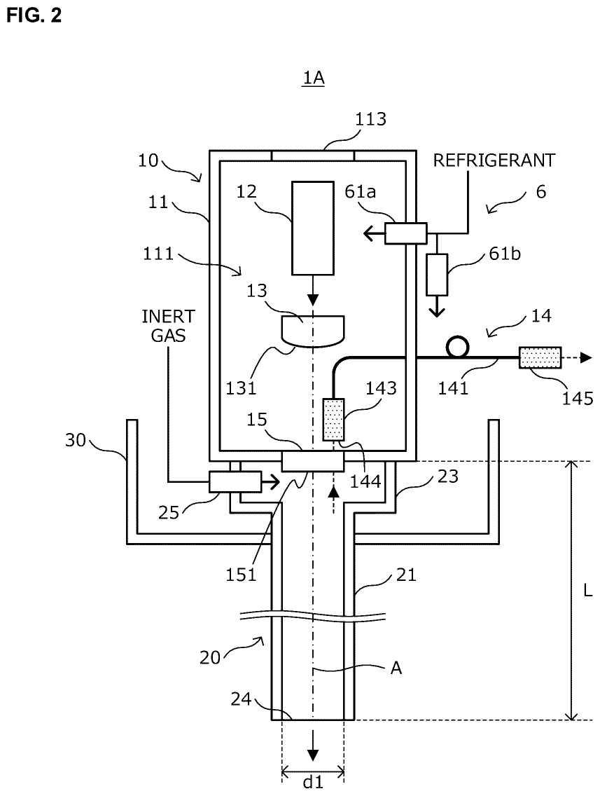

Next, a modification example of the optical device 1 for laser emission spectroscopic analysis of this embodiment will be described with reference to FIG. 2. FIG. 2 is an explanatory diagram schematically illustrating another example of the configuration of the optical device for laser emission spectroscopic analysis of this embodiment.

An optical device 1A for laser emission spectroscopic analysis of this modification example differs from the optical device 1 for laser emission spectroscopic analysis illustrated in FIG. 1 in that the condenser lens 13 is arranged in the casing 11. In the following, the explanation will focus on such differences, and similar matters will be omitted.

As described above, in the optical device 1A for laser emission spectroscopic analysis of this modification example, the condenser lens 13 is arranged on the laser oscillator 12 side than the light-receiving part 143 of the optical fiber light-receiver 14 in the casing 11. A light-guide window 15 is arranged on the cylindrical probe 20 side of the casing 11 instead of the condenser lens 13. An antireflection film of laser light is formed on each of the surface 131 of the condenser lens 13 on the object side and a surface 151 of the light-guide window 15 on the object side.

In the above configuration, the laser oscillation axis A of the laser oscillator 12 and the center axis of the cylindrical probe 20 are almost coaxial, and the surface normal direction of the light-receiving end surface 144 of the light-receiving part 143 of the optical fiber light receiver 14 is almost parallel to the laser oscillation axis A. This makes the optical device 1A for laser emission spectroscopic analysis lightweight and compact, similar to the optical device 1 for laser emission spectroscopic analysis described above.

### Laser Emission Spectroscopic Analyzer

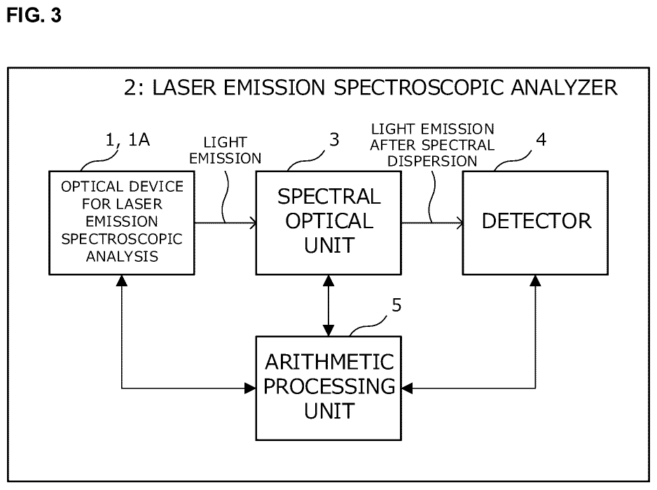

Next, a laser emission spectroscopic analyzer 2 having the optical device 1 for laser emission spectroscopic analysis or optical device 1A for laser emission spectroscopic analysis of this embodiment will be described in detail with reference to FIG. 3. FIG. 3 is a block diagram schematically illustrating an example of a configuration of a laser emission spectroscopic analyzer of this embodiment.

The laser emission spectroscopic analyzer 2 of this embodiment has the optical device 1 for laser emission spectroscopic analysis or optical device 1A for laser emission spectroscopic analysis, a spectral optical unit 3, a detector 4, and an arithmetic processing unit 5, as illustrated in FIG. 3. The laser emission spectroscopic analyzer 2 of this embodiment further preferably has the cooling mechanism 6, as schematically illustrated in FIG. 1 and FIG. 2.

The optical devices 1 and 1A for laser emission spectroscopic analysis have been described previously with reference to FIG. 1 and FIG. 2, so a detailed description is omitted hereafter.

The spectral optical unit 3 is connected to the optical fiber light receiver 14 (more precisely, the emitting part 145) in the optical device 1 or 1A for laser emission spectroscopic analysis, and spectrally disperses the light emission (plasma light) guided by the optical fiber light receiver 14. The spectral optical unit 3 is not limited, as long as it has enough resolution to disperse the light of each wavelength corresponding to the element to be analyzed (for example, at least Fe, Zn, and Al in the case of molten zinc). Various known spectral optical elements such as diffraction gratings and spectral prisms can be used. Various types of spectroscopes can also be used as the spectral optical unit 3. The plasma light is spectrally dispersed into different wavelengths by the spectral optical unit 3 and detected by the detector 4 located at a subsequent stage.

The detector 4 is a device that detects the light emission (plasma light) spectrally dispersed by the spectral optical unit 3, detects intensity of the light emission (plasma light) at each wavelength after the spectral dispersion, and outputs an electrical signal corresponding to such intensity. Examples of such detectors 4 include, for example, optical sensors such as CCD (charge coupled device), ICCD (image intensifier charge coupled device, image intensifier charge coupled device detector), CMOS (complementary metal oxide semiconductor), and PMT (photomultiplier tube).

Among the detectors mentioned above, the ICCD is more preferably used as the detector 4. The ICCD has a particularly high sensitivity among the detectors mentioned above. As described above, the optical fiber light receiver 14 receives the plasma light without being condensed and thus receives less plasma light than when the plasma light is condensed using a conventional optical member. However, the intensity of the spectrally dispersed plasma light can be detected with high precision even with such a small amount of light received by using the ICCD as the detector 4.

The detector 4 detects the intensity in a wavelength band including each wavelength corresponding to the target element of the object (for example, Fe, Zn, and Al in the case of molten zinc), and outputs the electrical signal corresponding to such intensity as detection data to the arithmetic processing unit 5 described below.

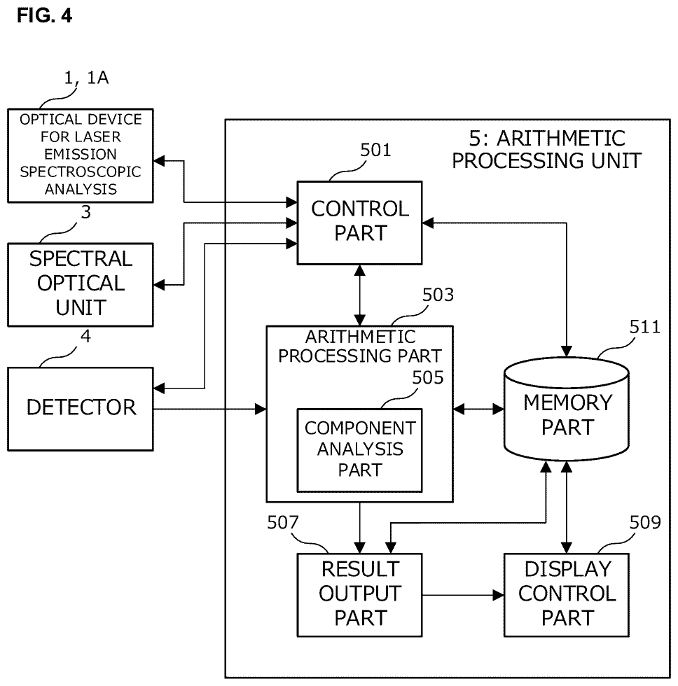

The arithmetic processing unit 5 is a device that comprehensively controls the operation of the optical device 1 or 1A for laser emission spectroscopic analysis, the spectral optical unit 3, and the detector 4 as described above, and performs spectroscopic analysis of an object based on the detection data output from the detector 4. In the following, such arithmetic processing unit 5 will be explained in detail with reference to FIG. 4. FIG. 4 is a block diagram illustrating an example of a configuration of an arithmetic processing unit held by the laser emission spectroscopic analyzer of this embodiment.

As illustrated in FIG. 4, the arithmetic processing unit 5 of this embodiment mainly includes a control part 501, an arithmetic processing part 503, a result output part 507, a display control part 509, and a memory part 511.

The control part 501 is achieved by, for example, a CPU (central processing unit), ROM (read only memory), RAM (random access memory), input device, output device, communication device, and other devices. The control part 501 is a processing part that comprehensively controls the functions of the optical device 1 or 1A for laser emission spectroscopic analysis of this embodiment, the spectral optical unit 3, and the detector 4. The control part 501 can also comprehensively control the functions of other mechanisms provided in the laser emission spectroscopic analyzer 2, such as the cooling mechanism 6 described below, for example.

In more detail, when starting analysis on an object, the control part 501 sends a control signal to the optical device 1 or 1A for laser emission spectroscopic analysis to start irradiation of laser light from the laser oscillator 12, and the laser oscillator 12 irradiates the laser light toward the object. The control part 501 also sends a trigger signal to the spectral optical unit 3 and the detector 4 to spectrally disperse the received plasma light and output the detection data regarding the intensity of each wavelength, and the detector 4 outputs the detection data regarding the plasma light to the arithmetic processing unit 5.

The arithmetic processing part 503 is achieved by, for example, a CPU, ROM, RAM, communication device, and other devices. The arithmetic processing part 503 is a processing part that obtains the detection data regarding the plasma light output from the detector 4 and performs various arithmetic processes on such detection data. This arithmetic processing part 503 has a component analysis part 505 as illustrated in FIG. 4.

The component analysis part 505 is achieved by, for example, a CPU, ROM, RAM, and other devices. The component analysis part 505 analyzes components of the object based on the detection results (that is, the detection data) of the plasma light by the detector 4.

In more detail, the component analysis part 505 performs component analysis based on the detection results (that is, the detection data) by the detector 4, for example, by LIBS. Concretely, the component analysis part 505 refers to the detection data to identify at which wavelength and at what intensity light was detected. The component analysis part 505 then identifies the component (element) where the light with the wavelength of interest is originated with reference to a database stored in the memory part 511 or the like. This allows the components contained in the object of interest to be identified.

The component analysis part 505 can also identify a content (concentration) of the identified components from the data on light-emission intensity contained in the obtained detection data. Such content may be calculated as a relative content from the light-emission intensity of each component identified as described above. In addition, a calibration curve showing the relationship between the light-emission intensity and content may be prepared in advance using a standard reagent or the like for the components contained in the object, and the content of each component may be calculated from the obtained light-emission intensity.

When the component analysis part 505 identifies the concrete components and their contents in the object as described above, it outputs the obtained results to the result output part 507 as analysis results. The component analysis part 505 may also store the obtained data on the analysis results in the memory part 511 as historical information after associating the data with time information on the date and time when the data was obtained.

The result output part 507 is achieved by, for example, a CPU, ROM, RAM, output device, communication device, and other devices. The result output part 507 outputs information on the components of the object of interest output from the arithmetic processing part 503 (more precisely, the component analysis part 505) to the user of the laser emission spectroscopic analyzer 2. Concretely, the result output part 507 outputs the data regarding the analysis results of the components output from the arithmetic processing part 503 to various servers and control devices or as paper media using a printer or other output devices, while associating the data with time data regarding the date and time when the data is generated. The result output part 507 may also output the data regarding the analysis results to various information processing devices such as external computers or various recording media.

The result output part 507 can output the data regarding the analysis results by the arithmetic processing part 503 to the display control part 509 described below.

The display control part 509 is achieved by, for example, a CPU, ROM, RAM, output device, communication device, and other devices. The display control part 509 performs display control when displaying the analysis results output from the result output part 507 on an output device such as a display held by the laser emission spectroscopic analyzer 2 or an output device provided outside the laser emission spectroscopic analyzer 2. This allows the user of the laser emission spectroscopic analyzer 2 to grasp the analysis results of the components of the object of interest on the spot.

The memory part 511 is an example of a memory device held by the laser emission spectroscopic analyzer 2 and is achieved, for example, by a ROM, RAM, storage device, and other devices. In this memory part 511, various parameters and process progress (for example, various data and databases stored in advance, programs, and the like) that need to be saved when the laser emission spectroscopic analyzer 2 of this embodiment performs some processing are recorded as appropriate. The control part 501, arithmetic processing part 503, component analysis part 505, result output part 507, display control part 509, and other parts can freely perform data read/write processing from/to the memory part 511.

The above is an example of the functions of the arithmetic processing unit 5 of this embodiment. Each of the above components may be formed by general-purpose members and circuits or may be formed by hardware specialized for the function of each component. In addition, a CPU or other devices may perform all the functions of each component. Therefore, it is possible to change the configuration to be used as appropriate according to the level of technology at the time of implementing this embodiment.

Computer programs to achieve each function of the arithmetic processing unit of this embodiment as described above can be produced and implemented in a personal computer, a process computer, which is a higher-level arithmetic processing unit, or other computers. A computer-readable recording medium in which such computer programs are stored can also be provided. The recording medium can be, for example, a magnetic disk, optical disk, magneto-optical disk, flash memory, or other media. The computer programs described above can also be distributed over a network, for example, without using a recording medium.

The cooling mechanism 6, which is preferably held by the laser emission spectroscopic analyzer 2 cools the devices in the casing unit 10, including the laser oscillator 12, with a refrigerant. For example, when cooled air is used as the refrigerant, the cooling mechanism 6 has a not-illustrated blower and blast pipes, and blow-out ports 61a and 61b as examples of refrigerant blow-out ports are provided at the ends of the blast pipes. As illustrated in FIG. 1 and FIG. 2, two types of blow-out ports are preferably provided as the blow-out ports: the blow-out port 61a, which supplies the supplied refrigerant to the inside of the casing 11, and the blow-out port 61b, which supplies the supplied refrigerant to the periphery of the casing 11.

The blow-out port 61a is attached to the casing 11, which allows the supply of refrigerant to the internal space 111 of the casing 11. The refrigerant supplied to the internal space 111 cools each device in the casing 11, especially the laser oscillator 12, and is discharged through the opening part 113. On the other hand, the blow-out port 61b supplies the refrigerant to a space between the hood 30 and the casing 11, for example. This allows the casing 11 to be cooled from the outside.

As a result, each device in the casing 11 is cooled by a double cooling mechanism, efficiently blocking heat from the object and efficiently cooling each device in the casing 11.

Although the above describes an air-cooled cooling mechanism, the present invention is not limited thereto. Various cooling mechanisms, such as liquid cooling like water cooling, and thermoelectric cooling using thermoelectric elements such as Peltier elements, may be employed as the cooling mechanism. A combination of multiple cooling mechanisms may also be used.

### Hardware Configuration of Arithmetic Processing Unit 5

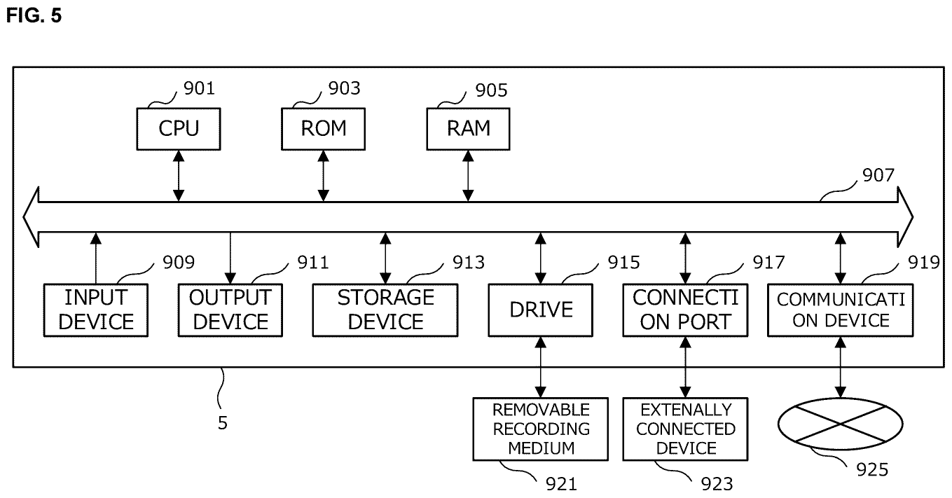

Next, a hardware configuration of the arithmetic processing unit 5 of this embodiment will be explained in detail with reference to FIG. 5. FIG. 5 is a block diagram for explaining the hardware configuration of the arithmetic processing unit 5 of this embodiment.

The arithmetic processing unit 5 mainly includes a CPU 901, a ROM 903, and a RAM 905. The arithmetic processing unit 5 further includes a bus 907, an input device 909, an output device 911, a storage device 913, a drive 915, a connection port 917, and a communication device 919.

The CPU 901 functions as a central processing unit and controller, and controls all or part of the operations in the arithmetic processing unit 5 according to various programs recorded in the ROM 903, RAM 905, storage device 913, or a removable recording medium 921. The ROM 903 stores programs, arithmetic parameters, and the like used by the CPU 901. The RAM 905 primarily stores programs used by the CPU 901 and parameters and the like that change from time to time in the execution of the programs. These are interconnected by the bus 907, which is formed by an internal bus such as a CPU bus.

The bus 907 is connected to an external bus, such as a PCI (peripheral component interconnect/interface) bus, through a bridge.

The input device 909 is an operating means operated by the user such as, for example, a mouse, keyboard, touch panel, buttons, switches, and levers. The input device 909 may be, for example, a remote control means (so-called a remote controller) using infrared rays or other radio waves, or an externally connected device 923 such as a PDA that is compatible with the operation of the arithmetic processing unit 5. Furthermore, the input device 909 is formed by, for example, an input control circuit and the like that generates input signals based on information entered by the user using the operation means described above and outputs them to the CPU 901. By operating this input device 909, the user can input various data and instruct processing operations to the arithmetic processing unit 5.

The output device 911 is formed by a device capable of visually or aurally notifying the user of the obtained information. Such devices include display devices such as CRT display devices, liquid crystal display devices, plasma display devices, EL display devices, and lamps, audio output devices such as speakers and headphones, printer devices, cell phones, facsimile machines, and other devices. The output device 911 outputs results obtained from various processes performed by the arithmetic processing unit 5, for example. Concretely, the display device displays the results obtained by the various processes performed by the arithmetic processing unit 5 in text or images. On the other hand, the audio output device converts audio signals made up of reproduced voice data, acoustic data, and other data, into analog signals and outputs them.

The storage device 913 is a device for storing data formed as an example of the memory part of the arithmetic processing unit 5. The storage device 913 is formed by, for example, a magnetic storage device such as an HDD (hard disk drive), semiconductor storage device, optical storage device, or magneto-optical storage device. This storage device 913 stores programs executed by the CPU 901 and various data, as well as various data obtained from external sources.

The drive 915 is a reader/writer for recording media and is built into or attached externally to the arithmetic processing unit 5. The drive 915 reads information recorded on the removable recording medium 921, such as an installed magnetic disk, optical disk, magneto-optical disk, or semiconductor memory, and outputs the information to the RAM 905. The drive 915 can also write records to the removable recording medium 921 such as an installed magnetic disk, optical disk, magneto-optical disk, or semiconductor memory. The removable recording medium 921 is, for example, a CD medium, DVD medium, Blu-ray (registered trademark) medium, or other media. The removable recording medium 921 may also be CompactFlash (registered trademark) (CF), a flash memory, SD memory card (secure digital memory card), or other media. The removable storage medium 921 may also be, for example, an IC card (integrated circuit card) equipped with a non-contact IC chip or electronic device.

The connection port 917 is a port for connecting devices directly to the arithmetic processing unit 5. Examples of the connection port 917 include a USB (universal serial bus) port, IEEE1394 port, SCSI (small computer system interface) port, RS-232C port, HDMI (registered trademark) (high-definition multimedia interface) port, and other ports. By connecting the external connection device 923 to the connection port 917, the arithmetic processing unit 5 directly obtains various data from the external connection device 923 or provides various data to the external connection device 923.

The communication device 919 is a communication interface formed by, for example, a communication device for connection to a communication network 925. The communication device 919 is, for example, a communication card or the like for wired or wireless LAN (local area network), Bluetooth (registered trademark), or WUSB (wireless USB). The communication device 919 may also be a router for optical communication, a router for ADSL (asymmetric digital subscriber line), or a modem for various types of communication, and other devices. This communication device 919 can send and receive signals and the like to and from the Internet and other communication devices in accordance with a predetermined protocol, such as TCP/IP, for example. The communication network 925 connected to the communication device 919 may be formed by a network or the like connected by wired or wireless means, and may be, for example, the Internet, a home LAN, an in-house LAN, infrared communication, radio wave communication, satellite communication, or other networks.

The above is an example of a hardware configuration that can achieve the functions of the arithmetic processing unit 5 according to the embodiment of the present invention. Each of the above components may be formed using general-purpose members or hardware specialized for the functions of each component. Therefore, it is possible to change the hardware configuration to be used as appropriate according to the level of technology at the time of implementing this embodiment.

### Molten Metal Plating Equipment

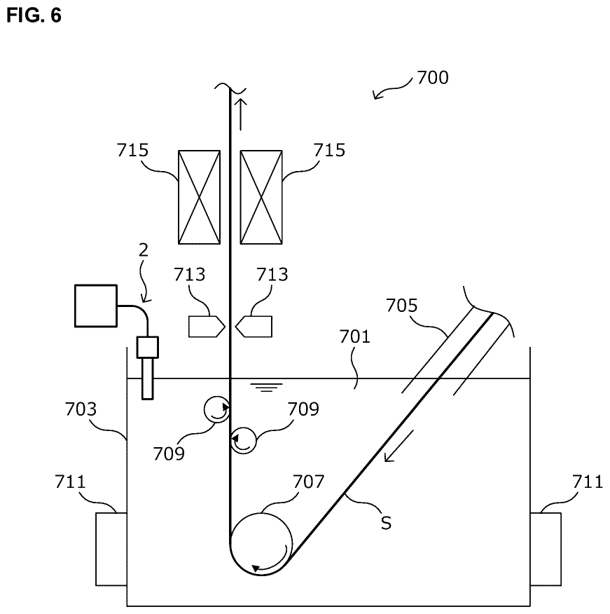

Next, an example of a molten metal plating equipment equipped with the laser emission spectroscopic analyzer described above will be described. FIG. 6 is a side view illustrating a schematic configuration of a hot-dip galvanizing apparatus of this embodiment. As an example of a molten metal bath, a hot-dip galvanizing bath 701 (hereinafter simply referred to as “plating bath”) in hot-dip galvanizing equipment 700 is described as a prototype. However, the present invention is not limited to such an example and can be applied to any other molten metal baths.

The hot-dip galvanizing equipment 700 is equipment for continuously depositing molten zinc on a surface of a steel strip S by immersing the steel strip S into the plating bath 701 filled with molten zinc. The hot-dip galvanizing equipment 700 includes a plating tank 703, a snout 705, an in-bath roll 707, a support roll 709, an inductor 711, a gas-wiping device 713, an alloying furnace 715, and the laser emission spectroscopic analyzer 2.

The plating tank 703 stores the plating bath 701 composed of molten zinc. In addition to Zn, the plating bath 701 of this embodiment contains, for example, about 0.12 to 0.15 mass % Al and about 0.02 to 0.1 mass % Fe. The temperature of the plating bath 701 is, for example, about 440 to 480° C. The snout 705 is arranged to be inclined so that one end of the snout is immersed in the plating bath 701. The in-bath roll 707 is located at the lowest position inside the plating tank 703. The in-bath roll 707 rotates along an arrow illustrated in the figure due to contact and shearing with the steel strip S.

The support rolls 709 are arranged inside the plating tank 703 on a downstream side of the in-bath roll 707 in a conveying direction of the steel strip S and are located to sandwich the steel strip S fed from the in-bath roll 707 from both sides. The support roll 709 is rotatably supported by a not-illustrated bearing (for example, sliding bearing, rolling bearing, and other bearings). Only one or three or more support rolls may be installed, or they may not be arranged.

The inductor 711 is an example of a heating device that heats the plating bath 701 filled in the plating tank 703. As illustrated in FIG. 6, the inductors 711 of this embodiment are provided in plural on side wall portions of the plating bath 703 to control the bath temperature of the plating bath 701. A heating means for heating the plating bath 701 is not limited to the inductor 711, and any known technology can be used.

The gas-wiping device 713 is arranged above the plating tank 703 and has a function of controlling a deposition amount of molten metal by spraying gas (for example, nitrogen, air) onto surfaces of both sides of the steel strip S to scrape off molten metal deposited on the surface of the steel strip S.

The alloying furnace 715 is an example of a heating device that heats the steel strip S to a predetermined temperature after the gas wiping. The alloying furnace 715 raises the temperature of the steel strip S by heating to promote alloying of a plating layer of molten metal deposited on the surface of the steel strip S. As the alloying furnace 715, known technologies such as induction heaters, for example, are used.

The steel strip S annealed in the annealing furnace, which is an upstream process, is immersed in the plating tank 703 filled with the plating bath 701 through the snout 705, pulled up in a vertical direction while passing through the in-bath roll 707 and support roll 709, and conveyed outside the plating bath 701. The steel strip S conveyed outside the plating bath 701 passes through the alloying furnace 715 after weight of the molten metal deposited on the surface is adjusted by the gas-wiping device 713.

The laser emission spectroscopic analyzer 2 is a device that has a function of detecting and analyzing each component present in the plating bath 701. The laser emission spectroscopic analyzer 2 quantifies, for example, contents of Fe and Al from signal strength data of the target elements obtained by irradiating a pulse laser while supplying inert gas in the plating bath 701. In other words, the laser emission spectroscopic analyzer 2 of this embodiment has a configuration for performing the LIBS method using the plating bath of molten zinc as the measurement object.

The above is an example of a hot-dip galvanizing equipment to which the laser emission spectroscopic analyzer of the present invention is applied.

### Laser Emission Spectroscopic Analysis Method

Next, a laser emission spectroscopic analysis method of this embodiment will be described. The laser emission spectroscopic analysis method of this embodiment is a method of analyzing molten metal in the plating bath of the molten metal plating using the laser emission spectroscopic analyzer of the present invention.

In the following, it is described the case when the laser emission spectroscopic analyzer 2 equipped with the optical device 1 for laser emission spectroscopic analysis described above is used to analyze the hot-dip galvanizing in the hot-dip galvanizing equipment 700 described above as the molten metal plating, as an example.

First, the laser oscillator 12 in the casing unit 10 of the optical device 1 for laser emission spectroscopic analysis oscillates the laser light under the control of the arithmetic processing unit 5. The oscillated laser light is condensed by the condenser lens 13 and guided by the cylindrical probe 20 to be focused near the opening end 24. The inert gas such as Ar, for example, is supplied from the gas inlet 51 of the cylindrical probe 20 toward the opening end 24. The laser light is then irradiated onto the object, the molten metal (Fe, Zn, Al, and other metals) in the plating bath 701. As a result, plasma is generated at the interface between the inert gas and the molten metal, and plasma light is generated accordingly.

The generated plasma light is guided by the cylindrical probe 20, and a part of the light is received by the optical fiber light receiver 14 (more concretely, the light-receiving end surface 144 of the light-receiving part 143) in the casing unit 10 without being condensed. The received plasma light is guided through the bundle fiber 141 and the emitting part 145 to the spectral optical unit 3. The spectral optical unit 3 spectrally disperses the guided plasma light into various wavelengths under the control of the arithmetic processing unit 5 and the spectrally dispersed plasma light reaches the detector 4 at a subsequent stage. The detector 4 detects the spectrally dispersed plasma light at each wavelength under the control of the arithmetic processing unit 5, and measures the intensity of the plasma light at each wavelength. The detector 4 then outputs an electrical signal corresponding to the intensity of the plasma light as measurement data to the arithmetic processing unit 5. As a result, the intensity of plasma light originating from each component such as Fe, Zn, and Al contained in the plating bath 701 is identified.

The component analysis part 505 in the arithmetic processing unit 5 analyzes the content (concentration) of each component such as Fe, Zn, and Al using the measurement data including the electrical signal corresponding to the intensity of the plasma light originating from each component such as Fe, Zn, and Al as described above, by known methods. This makes it possible to determine the content (concentration) of each component such as Fe, Zn, and Al in the plating bath 701 of interest.

In this case, for example, hot-dip galvanizing with different Al concentrations was measured by the analysis method of this embodiment to obtain a light-emission intensity ratio I(Al)/I(Zn) between Al and Zn and a part of molten zinc of each Al concentration is sampled, acid dissolved, and quantified by an ICP light-emission analysis method or other methods, thereby creating in advance a calibration curve showing a relationship between the Al concentration and the light-emission intensity ratio, and storing the calibration curve in the memory part 511. The component analysis part 505 can convert the light-emission intensity ratio I(Al)/I(Zn) calculated from the measurement data to the Al concentration in molten zinc using the obtained measurement data and the calibration curve.

In this embodiment, the laser emission spectroscopic analyzer 2 has the above configuration, so that a part of the generated plasma light is received by the optical fiber light receiver 14 without being condensed, and the light of each wavelength corresponding to each component of the molten metal such as Fe, Zn, and Al is detected by the detector 4. This allows the influence on the analysis results due to thermal distortion, vibration, and bath level fluctuation of the hot-dip galvanizing equipment 700 to be suppressed, and enables analysis of each component with relatively high precision over a long period of time.

Furthermore, the optical device 1 for laser emission spectroscopic analysis held by the laser emission spectroscopic analyzer 2 can omit optical members for condensing the plasma light and adjusting the oscillation axis of the laser light owing to the above configuration and is much smaller and lighter than the conventional optical device for laser emission spectroscopic analysis. This enables analysis in narrow spaces where the conventional optical device cannot be located and at different measurement points.

### EXAMPLES

In the following, the optical device for laser emission spectroscopic analysis, laser emission spectroscopic analyzer, laser emission spectroscopic analysis method, and molten metal plating equipment of this embodiment will be described with concrete examples.

First, an optical device for laser emission spectroscopic analysis corresponding to the optical device 1 for laser emission spectroscopic analysis illustrated in FIG. 1 was fabricated. A concrete device configuration and measurement conditions are as follows.

### Laser

Laser oscillator: Viron manufactured by LUMIBIRD (diode-pumped Nd: YAG laser)

Laser oscillation conditions: 1064 nm wavelength, 20 Hz, 50 mJ/pulse (Condenser lens)

Focal length: 900 mm

A surface of the condenser lens on the laser oscillator side was provided with an antireflection film corresponding to the wavelength of the laser light.

### Optical Fiber Light-Receiver

Optical fiber (manufactured by Mitsubishi Cable Corporation, Type 1 standard fiber 15 m (32 cores))

### Cooling Mechanism

Air-cooling system using compressed air

### Spectral Optical Unit and Detector

The following spectroscope was used as the spectral optical unit and detector.

Spectroscope: Double grating spectrograph (NP250-2) manufactured by SOL instruments, TOP: 600 lines/mm, BOTTOM: 1200 lines/mm, slit width: 30 μm

Detector: ICCD camera manufactured by ANDOR Co., Ltd. (istar, gain: 2000, gate width: 10000 ns, delay: 1000 ns, integration frequency: 1 time, repetition frequency: 1 time)

### Cylindrical Probe

Length of cylindrical probe: 1000 mm

Probe material: Sialon (silicon nitride-base)

Pure Ar gas with a purity of 99.9999% or higher was used as the inert gas and was supplied to the cylindrical probe at a flow rate of 1.0 L/min.

In the above configuration, the laser oscillation axis and the center axis of the cylindrical probe were set to be parallel to each other, and the surface normal direction at the light-receiving end surface of the optical fiber light receiver and the laser oscillation axis were also set to be parallel to each other.

The size of the casing of the optical device for laser emission spectroscopic analysis fabricated as described above was 330 mm (width)×220 mm (height)×200 mm (depth). In the case of the conventional configuration in which a mirror for optical axis adjustment was arranged, the size of about 500 mm (width)×250 mm (height)×640 mm (depth) was the limit for miniaturization. The fabricated optical device for laser emission spectroscopic analysis can be significantly reduced in size and weight.

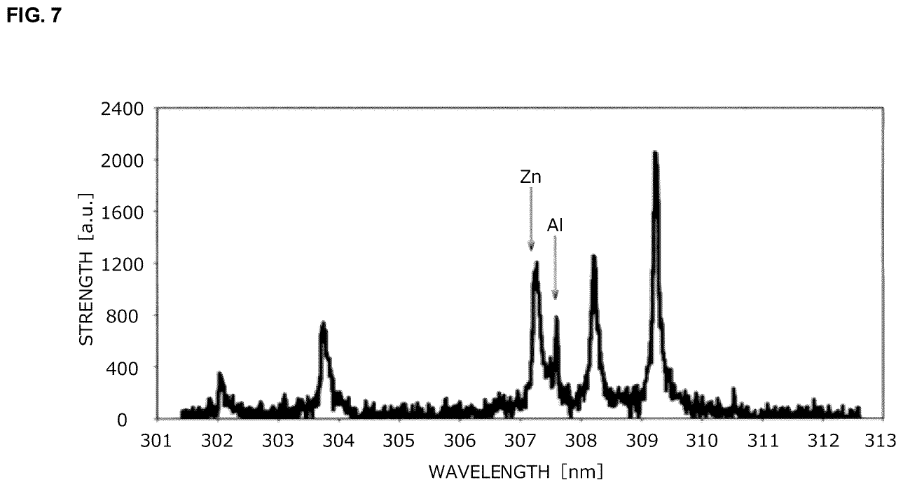

A laser emission spectroscopic analyzer was configured using the fabricated optical device for laser emission spectroscopic analysis, and the spectral intensity of Zn and Al in the molten zinc bath over time was measured using an integration time of 300 seconds for the detected signals (signal strength). FIG. 7 illustrates the laser-induced breakdown spectrum (LIBS spectrum) of the molten zinc bath measured under the above conditions.

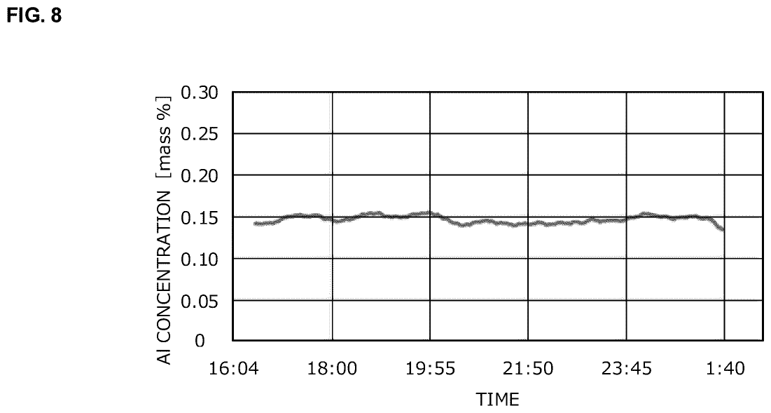

The time course of the spectrum illustrated in FIG. 7, where the signal at 307.6 nm corresponding to the light-emission wavelength of Al is normalized by the signal at 307.3 nm corresponding to the light-emission wavelength of Zn, is illustrated in FIG. 8.

The Al concentration in the measured molten zinc bath was not intentionally changed and can be assumed to be constant over time. As illustrated in FIG. 8, a peak ratio (signal strength ratio) of Al/Zn was almost constant. In summary, it was shown that the laser emission spectroscopic analyzer of this embodiment can precisely quantify the components of the molten metal over time.

On the other hand, when the same experiment was conducted using an optical device for laser emission spectroscopic analysis where plasma light is incident on a light receiver by using a lens for condensing the plasma light, the Al/Zn peak ratio (signal strength ratio) varied about twice as much as that of the present invention where the light is not condensed as described above, during about one day of measurement. This suggests that the present configuration, in which the plasma light is received without being condensed, rather improves the precision of the measurement.

Preferred embodiments of the present invention have been described above in detail with reference to the attached drawings, but the present invention is not limited to the embodiments. It should be understood that various changes and modifications are readily apparent to those skilled in the art who have the common general knowledge in the technical field to which the present invention pertains, within the scope of the technical spirit as set forth in claims, and they should also be covered by the technical scope of the present invention.

The embodiments disclosed herein are in all respects illustrative and not restrictive. The above embodiments may be omitted, substituted, or modified in various forms without departing from the scope of the appended claims, the configurations within the technical scope of the invention as described below, and the main idea thereof. For example, constituent elements of the above embodiments may be arbitrarily combined to the extent that the effects thereof are not impaired. In addition, from such arbitrary combination, the action and effect for each of the constituent elements for the combination will naturally be obtained, as well as other actions and other effects that are obvious to those skilled in the art from the description herein.

The effects described herein are only illustrative or exemplary, not limiting. In other words, the technology for the present invention can produce other effects that are obvious to those skilled in the art from the description herein, either together with or in place of the above effects.

### EXPLANATION OF CODES

1, 1A optical device for laser emission spectroscopic analysis
2 laser emission spectroscopic analyzer
3 spectral optical unit
4 detector
5 arithmetic processing unit
6 cooling mechanism
10 casing unit
11 casing
12 laser oscillator
13 condenser lens
14 optical fiber light receiver
20 cylindrical probe
21 probe part
23 base end part
24 opening end
25 gas inlet
30 hood
61a, 61b refrigerant blow-out port
111 internal space
113 opening part
141 bundle fiber
143 light-receiving part
145 emitting part
144 light-receiving end surface
501 control part
503 arithmetic processing part
505 component analysis part
507 result output part
509 display control part
511 memory part
700 hot-dip galvanizing equipment
701 plating bath
703 plating tank
705 snout
707 in-bath roll
709 support roll
711 inductor
713 gas-wiping device
715 alloying furnace
S steel strip

1. An optical device for laser emission spectroscopic analysis, which is used for analyzing a component of molten metal, comprising:
a casing unit that has a laser oscillator oscillating laser light, a condenser lens condensing the laser light and on which the laser light emitted from the laser oscillator is directly incident, and an optical fiber light receiver receiving light emitted from plasma generated by irradiating the laser light onto the molten metal at a light-receiving end surface; and
a cylindrical probe that is connected to the casing unit so that a center axis is parallel to an oscillation axis of the laser light in the laser oscillator, supplies inert gas to an opening end located downstream of the laser light traveling direction and guides the laser light toward the opening end to irradiate on the molten metal, wherein
a surface normal direction at the light-receiving end surface of the optical fiber light receiver is parallel to the oscillation axis of the laser light.

2. The optical device for laser emission spectroscopic analysis according to claim 1, wherein
the cylindrical probe is connected to the casing unit so that the center axis is coaxial with the oscillation axis of the laser light.

3. The optical device for laser emission spectroscopic analysis according to claim 1, wherein
the condenser lens is provided at a connection part between the casing unit and the cylindrical probe.

4. The optical device for laser emission spectroscopic analysis according to claim 1, wherein
at least a part of the light emission is incident on the light-receiving end surface of the optical fiber light receiver in a state where the light is not condensed.

5. The optical device for laser emission spectroscopic analysis according to claim 1, wherein
the laser oscillator is a diode-pumped laser oscillator.

6. The optical device for laser emission spectroscopic analysis according to claim 1, wherein
the condenser lens has an antireflection film on its surface to prevent reflection of the laser light.

7. The optical device for laser emission spectroscopic analysis according to claim 1, further comprising:
an angle adjustment mechanism that adjusts a lens optical axis direction of the condenser lens by changing an attachment angle of the condenser lens.

8. A laser emission spectroscopic analyzer, comprising:
the optical device for laser emission spectroscopic analysis according to claim 1;
a spectral optical unit that spectrally disperses the light emission guided by the optical fiber light receiver;
a detector that detects the light emission spectrally dispersed by the spectral optical unit; and
a component analysis unit that analyzes components of molten metal based on detection results of the light emission by the detector.

9. The laser emission spectroscopic analyzer according to claim 8, wherein
the detector is an image intensifier charge-coupled device detector.

10. The laser emission spectroscopic analyzer according to claim 8, further comprising:
a cooling mechanism that cools an inside of the casing unit.

11. A laser emission spectroscopic analysis method, which analyzes molten metal in a plating bath for molten metal plating by using the laser emission spectroscopic analyzer according to claim 8.

12. The laser emission spectroscopic analysis method according to claim 11, wherein
the molten metal plating is hot-dip galvanizing.

13. A molten metal plating equipment, comprising:
the laser emission spectroscopic analyzer according to claim 8.

14. The molten metal plating equipment according to claim 13, which is a hot-dip galvanizing equipment to apply hot-dip galvanizing.
